(19) United States
(12) Patent Application Publication
(10) Pub. No.: US 2024/0408118 A1
(43) Pub. Date: Dec. 12, 2024

(54) AN OPHTHALMIC FORMULATION FOR TREATMENT AND PREVENTION OF CATARACT AND PRODUCTION METHOD THEREOF

(71) Applicant: VSY BIYOTEKNOLOJI VE ILAC SANAYI ANONIM SIRKETI, Istanbul (TR)

(72) Inventors: Efe CAN, Istanbul (TR); Faruk OYTUN, Istanbul (TR)

(73) Assignee: VSY BIYOTEKNOLOJI VE ILAC SANAYI ANONIM SIRKETI, Istanbul (TR)

(21) Appl. No.: 18/702,377

(22) PCT Filed: Dec. 31, 2021
(86) PCT No.: PCT/TR2021/051679

§ 371 Date: Apr. 18, 2024

(30) Foreign Application Priority Data
Oct. 18, 2021 (TR) 2021/016195

(51) Int. Cl. A61K 31/661; A61K 31/145; A61K 33/30; A61K 47/36

(52) U.S. Cl. CPC A61K 31/661; A61K 31/145; A61K 33/30; A61K 47/36

(57) ABSTRACT

An ophthalmic formulations developed for the treatment and prevention of cataract and a method for preparation thereof are provided. The objective of the present invention is to develop an ophthalmic topical formulation which the patients can use themselves non-invasively in order to prevent development of cataract in risk groups or to treat existing cataracts.

### CROSS REFERENCE TO THE RELATED APPLICATIONS

This application is the national phase entry of International Application No. PCT/TR2021/051679, filed on Dec. 31, 2021, which is based upon and claims priority to Turkish Patent Application No. 2021/016195, filed on Oct. 18, 2021, the entire contents of which are incorporated herein by reference.

### TECHNICAL FIELD

The present invention relates to ophthalmic formulations developed for the treatment and prevention of cataract and a method for preparation thereof.

### BACKGROUND

Cataract is the leading cause of blindness worldwide. Low vision and blindness significantly reduce the quality of life and increase the labor loss. Cataract is the blurring of vision due to the lens, which is in the eye and functions so that the light coming from outside can reach the visual center clearly, losing its transparency and becoming opaque, and condensing on the lens.

Although the most commonly known type of cataract is age-related (in people aged 55-60 on average and older), there are also types of cataracts that are present from the birth of the person or due to medications the person takes during their lifetime, blows to the eye and metabolic disorders such as diabetes. In the process of cataract formation, the patient initially complains of visual disturbances such as experiencing difficulties in the vividness of colors, hypersensitivity to light, visual difficulties in slightly dark environments, and inability to clearly see far or near; however, as the opacity increases, the difficulties experienced in seeing far and near begin to affect the daily life of the patient negatively.

In the current art, the treatment process of cataract is generally based on the removal of the eye lens that has lost its transparency by a surgical process and the replacement thereof with an intraocular lens. Since it is not possible to treat cataract with medication or glasses, it is essential that patients undergo an operation to return to their daily lives. Although these operations (called extracapsular extraction or extracapsular surgery) used to be an operation in which the lens was removed through a large incision and it was finished with a suture, over time it has started to be performed with small incisions and without stitches. The most up-to-date surgical system in the state of the art is a surgical method that is called phacoemulsification (also known as laser cataract surgery, stitch-free and phaco), wherein the cataract is dissolved in the eye by means of a phaco device which vibrates 40,000 times per second and generates sound waves. In this way, after the cataract is extracted and removed from the eye, the foldable lens made of acrylic material is positioned inside the eye. After this surgery, which is performed in a short time like half an hour, the patient can return to their daily life after 2-3 days. However, this and other currently applied cataract operations are high cost surgical processes as well as they may cause many long and short term complications in the patient. Furthermore, access to this treatment is still limited, especially in developing countries, as competent physicians and well-equipped centers are required for surgery.

Chinese patent document no CN105879007, an application known in the state of the art, relates to an eye drop for cataract treatment and a preparation method in which the active component of the eye drop has a considerable synergistic effect.

International patent document no WO2020152527, an application known in the state of the art, relates to a liquid composition for treatment of eyes. The said invention relates to ophthalmic pharmaceutical compositions which treats development of cataract in the eye.

Chinese patent document no CN102176920, an application known in the state of the art, relates to ophthalmic solution composition, and it discloses eye drop or eye gel comprising natural curcuminoids.

Australian patent document no AU2006265248, an application known in the state of the art, relates to a drug in drop form for the treatment of corneal diseases.

Chinese patent document no CN1899304, an application known in the state of the art, discloses an eye drop for cataract and a preparation method thereof.

Mexican patent document no MX2014015124, an application known in the state of the art, discloses a pharmaceutical composition comprising zinc, niacin, cocarboxylase, vitamin C, hyaluronic acid, glutathione, magnesium, chromium and selenium and developed for ophthalmic use.

### SUMMARY

The objective of the present invention is to develop an ophthalmic topical formulation which the patients can use themselves non-invasively in order to prevent development of cataract in risk groups or to treat existing cataracts.

### DETAILED DESCRIPTION OF THE EMBODIMENTS

The ophthalmic formulation according to the present invention is prepared in phosphate buffer or non-phosphate buffer solution, and comprises by weight
linear and/or crosslinked forms of sodium hyaluronate in range of 0.01%-5%,
osmoprotectants in range of 0.001%-5%,
vitamin in range of 0.001%-5%,
amino acids in range of 0.001%-5%,
antioxidant in range of 0.001%-5%,
mineral salt in range of 0.001%-5%,
phospholipids in range of 0.001%-5%,
electrolytes in range of 0.001%-5%, and
drug delivery systems in range of 0.001%-5%.

Within the scope of the invention, linear and/or crosslinked forms of sodium hyaluronate with a molecular weight of 1000 Da-7 MDa are used, and sodium hyaluronate with low and high molecular weight is important in terms of determining the viscosity, penetration and drug release properties of the final product. Linear-linked forms and crosslinked forms of sodium hyaluronate can be used both together and separately within the scope of the invention. Its linear form allows the drop to spread over the entire ocular surface, while its crosslinked form allows the drop to remain on the ocular surface for a longer time.

Osmoprotectants are compounds which can penetrate through both sides of the double-layered lipid and provide osmotic balance and thereby protecting the cell from damage due to osmotic pressure. Especially in glucose-induced cataract formation, lens epithelial cells and lens fibrils are subjected to osmotic stress. Osmoprotectants enable the lens to maintain its natural metabolism by means of neutralizing osmotic stress. In one embodiment of the invention, the osmoprotectants in the formulation according to the invention are selected from the group comprising L-carnitine, betaine, putrescine, spermidine, spermine, glycinebetaine, b-alanine betaine, choline-O-sulfate, dimethyl-sulfonio propionate, trehalose, erythrole, fructan, mannitol, dextran, sorbitol, proline, ectoin and combinations thereof.

Oxidative stress has an important role in the onset and development of cataracts. Oxidative byproducts of cellular metabolism and ultraviolet radiation cause damage in many structural elements of the lens and initiate cataract development. Increasing reactive oxygen derivatives causes disulfide-linked dimer formation or precipitation of critical enzymes and membrane proteins, as well as lipid peroxidation, thereby initiating cataract formation. Vitamins (vitamins C and E) and carotenoids (vitamin A and its derivatives), which are among important non-enzymatic antioxidative agents, have beneficial effects on the lens maintaining its structural integrity and function. Dietary intake of certain vitamins and carotenoids in high doses is known to significantly reduce the development of age-related cataract. Lutein, zeaxanthin, vitamin E/C and carotenoids are among the main antioxidants used to prevent cataract. Glutathione, which is found abundantly in the lens, has a vital role in preventing oxidative stress. Increasing concentrations of glutathione and the enzyme glutathione synthase are known to inhibit cataract formation. Glutathione and L-cysteine protect the lens from free oxygen species and lipid peroxidation. In one embodiment of the invention, the antioxidants in the formulation according to the invention are selected from a group comprising glutathione, allicin, astaxanthin, N-Acetylcarnosine (NAC), epigallocatechin gallate (EGCG), coenzyme Q10 (CoQ10), curcumin, polyphenols, quercetin, alpha lipoic acid, resveratrol, alpha tocopherol, pyruvate, carotene, beta carotene, trolox, hydroxytyrosol, tyrosol, ferulic acid, caffeic acid, rutin, diosmin, melatonin, taurine, hypotaurine, and combinations thereof. In one embodiment of the invention, the vitamins in the formulation according to the invention are selected from the group comprising vitamin A derivatives such as retinal, retinol, pro-vitamin A, retinoic acid, vitamin B derivatives such as vitamin B1 (thiamine), vitamin B2 (riboflavin), vitamin B3 (nicotinamide), vitamin B5 (pantothenic acid), vitamin B6 (pyridoxine), vitamin B8 (biotin), vitamin B9 (folacin), vitamin B12 (cobalamins), vitamin C derivatives such as L-ascorbic acid, tetrahexyldecyl ascorbate, ascorbyl glucoside, ethylated ascorbic acid, ascorbyl palmitate, magnesium ascorbyl palmitate, magnesium ascorbyl phosphate, calcium ascorbate, sodium ascorbate, and sodium ascorbyl phosphate, vitamin D and vitamin K derivatives, and combinations thereof.

Deficiency of certain minerals, such as zinc, plays a critical role in aging-related cataract. Zinc metalloenzyme activity in lenses with cataract is lower than in normal lenses. Furthermore, many minerals act as cofactors for the enzymatic activities required for maintaining the normal course of lens metabolism and antioxidant balance. In one embodiment of the invention, the mineral salts in the formulation according to the invention are selected from the group comprising zinc sulfate, zinc acetate, zinc glutamate, zinc PCA, calcium chloride, calcium carbonate and calcium phosphate, tricalcium citrate, calcium lactate, calcium lactate gluconate, calcium gluconate, magnesium oxide, magnesium citrate, magnesium gluconate, magnesium chloride, magnesium sulfate, magnesium lactate, magnesium aspartate hydrochloride, potassium chloride, potassium carbonate, selenium, lactate, citrate, borate, and combinations thereof.

In an embodiment of the present invention, in order to maintain the natural electrolyte balance of the aqueous humor, the electrolytes in the formulation according to the present invention are selected from the group comprising potassium, bicarbonate, sodium, chlorine, magnesium, manganese, calcium, and combinations thereof.

Decreased amino acid levels are seen especially in the glucose-induced cataract form. Taurine concentration is considerably decreased in lenses with cataract. Taurine also acts as an organic osmolyte. In addition, taurine exhibits its antioxidant effects by binding free oxidant metalloproteins. Along with all these effects, taurine also inhibits cataract development. As a natural antioxidant, L-carnosine neutralizes hydroxyl radicals and superoxide radicals, and it also inhibits lipid peroxidation. With these effects, it prevents the development of cataract by protecting the crystalline lens from damage caused by oxidative stress. In one embodiment of the invention, the amino acids in the formulation according to the present invention are selected from the group comprising tryptophan, taurine, proline, cystine, asparagine, histidine and combinations thereof.

It is known that lipid oxidation leads to a significant loss of unsaturated phospholipids, including phosphatidylcholine, and the relative abundance of dihydrosphingomyelin during age-related cataract. Since the fibers of the human lens rely on phospholipids for their integrity, decrease in phospholipid levels can lead to cataract formation. This suggests the possibility that phospholipids may stop or slow down the progression of age-related cataract. In one embodiment of the invention, the phospholipids in the formulation according to the present invention are selected from a group comprising citicoline and other phosphatidyl cholines and combinations thereof.

The cornea has a strong hydrophobic structure. There are tight junctions between corneal epithelial cells called desmosomes and hemi-desmosomes. This structure of the cornea is the most important barrier for adequate penetration of aqueous solutions into the anterior chamber. Drug delivery systems are needed to achieve therapeutic concentrations in the aqueous and crystalline lens without reaching toxic concentrations at the ocular surface. In one embodiment of the invention, the drug delivery systems in the formulation according to the present invention are selected from a group comprising liposomes, niosomes, nanoparticles, dendrimers, micelles, polymer-drug conjugates and combinations thereof.

In one embodiment of the invention, the formulation is used in disposable topical vials, multi-use topical preservative-free bottles or in gel form. Contrary to the current method, with the ophthalmic topical formulation that can be used by everyone everywhere, non-invasively, it is aimed to treat the existing cataract, and also to prevent the development of cataract in risk groups.

In one embodiment of the invention, the formulation according to the present invention is used for the treatment and prevention of cataracts. The patient should use it 2 times a day by dropping. It will be used for the treatment and prevention of cataract.

The production method of the formulation according to the present invention comprises the steps of
Preparing buffer solution,
Filtering the buffer solution through 0.2 micron,
Adjusting the pH of the buffer solution to the value range of 6.8-7.6,
Mixing the components to be used in the formulation in the buffer solution with the help of a mixer at 40-250 rpm for 1-10 hours,
Degassing the mixture with vacuum,
Filtering the mixture through 0.2 micron filters,
Filling the mixture into vials and bottles under sterile conditions,
Labelling and packing the final products.

What is claimed is:

1. An ophthalmic formulation prepared in a phosphate buffer or non-phosphate buffer solution, comprising by weight
linear and/or crosslinked forms of sodium hyaluronate in a range of 0.01%-5%,
an osmoprotectant in a range of 0.001%-5%,
a vitamin in a range of 0.001%-5%,
an amino acid in a range of 0.001%-5%,
an antioxidant in a range of 0.001%-5%,
a mineral salt in a range of 0.001%-5%,
a phospholipid in a range of 0.001%-5%,
an electrolyte in a range of 0.001%-5%, and
a drug delivery system in a range of 0.001%-5%.

2. The ophthalmic formulation according to claim 1, wherein the osmoprotectant is at least one selected from the group consisting of L-carnitine, betaine, putrescine, spermidine, spermine, glycinebetaine, b-alanine betaine, choline-O-sulfate, dimethyl-sulfonio propionate, trehalose, erythrole, fructan, mannitol, dextran, sorbitol, proline, and ectoin.

3. The ophthalmic formulation according to claim 1, wherein the antioxidant is at least one selected from the group consisting of glutathione, allicin, astaxanthin, N-Acetylcarnosine (NAC), epigallocatechin gallate (EGCG), coenzyme Q10 (CoQ10), curcumin, polyphenols, quercetin, alpha lipoic acid, resveratrol, alpha tocopherol, pyruvate, carotene, beta carotene, trolox, hydroxytyrosol tyrosol, ferulic acid, caffeic acid, rutin, diosmin, melatonin, taurine, and hypotaurine.

4. The ophthalmic formulation according to claim 1, wherein the vitamin is at least one selected from the group consisting of vitamin A derivatives, vitamin B derivatives, vitamin C derivatives, vitamin D derivatives, and vitamin K derivatives, the vitamin A derivatives comprise retinal, retinol, pro-vitamin A, and retinoic acid, the vitamin B derivatives comprise vitamin B1 (thiamine), vitamin B2 (riboflavin), vitamin B3 (nicotinamide), vitamin B5 (pantothenic acid), vitamin B6 (pyridoxine), vitamin B8 (biotin), vitamin B9 (folacin), and vitamin B12 (cobalamins), and the vitamin C derivatives comprise L-ascorbic acid, tetrahexyldecyl ascorbate, ascorbyl glucoside, ethylated ascorbic acid, ascorbyl palmitate, magnesium ascorbyl palmitate, magnesium ascorbyl phosphate, calcium ascorbate, sodium ascorbate, and sodium ascorbyl phosphate.

5. The ophthalmic formulation according to claim 1, wherein the mineral salt is at least one selected from the group consisting of zinc sulfate, zinc acetate, zinc glutamate, zinc PCA, calcium chloride, calcium carbonate, calcium phosphate, tricalcium citrate, calcium lactate, calcium lactate gluconate, calcium gluconate, magnesium oxide, magnesium citrate, magnesium gluconate, magnesium chloride, magnesium sulfate, magnesium lactate, magnesium aspartate hydrochloride, potassium chloride, potassium carbonate, selenium, lactate, citrate, and borate.

6. The ophthalmic formulation according to claim 1, wherein the electrolyte is at least one selected from the group consisting of potassium, bicarbonate, sodium, chlorine, magnesium, manganese, and calcium.

7. The ophthalmic formulation according to claim 1, wherein the amino acid is at least one selected from the group consisting of tryptophan, taurine, proline, cystine, asparagine, and histidine.

8. The ophthalmic formulation according to claim 1, wherein the phospholipid is at least one selected from the group consisting of citicoline and other phosphatidyl cholines thereof.

9. The ophthalmic formulation according to claim 1, wherein the drug delivery system is at least one selected from the group consisting of a liposome, a noisome, a nanoparticle, a dendrimer, a micelle, and a polymer-drug conjugate.

10. A method of using the ophthalmic formulation according to claim 1 in a treatment and a prevention of cataract.

11. The ophthalmic formulation according to claim 1, wherein the ophthalmic formulation is in disposable topical vials, reusable topical preservative-free vials, or in a gel form.

12. A production method of the ophthalmic formulation according to claim 1, comprising steps of
preparing a buffer solution,
filtering the buffer solution through a first 0.2 micron filter to obtain a filtered buffer solution,
adjusting a pH of the filtered buffer solution to a value range of 6.8-7.6_to obtain an adjusted buffer solution,
mixing components to be used in the ophthalmic formulation in the adjusted buffer solution with a help of a mixer at 40-250 rpm for 1-10 hours to obtain a mixture,
degassing the mixture with a vacuum to obtain a degassed mixture,
filtering the degassed mixture through a second 0.2 micron filter to obtain a filtered mixture,
filling the filtered mixture into vials and bottles under a sterile condition to obtain final products,
labelling and packing the final products.

13. A method of using the ophthalmic formulation according to claim 2 in a treatment and a prevention of cataract.

14. A method of using the ophthalmic formulation according to claim 3 in a treatment and a prevention of cataract.

15. A method of using the ophthalmic formulation according to claim 4 in a treatment and a prevention of cataract.

16. A method of using the ophthalmic formulation according to claim 5 in a treatment and a prevention of cataract.

17. A method of using the ophthalmic formulation according to claim 6 in a treatment and a prevention of cataract.

18. A method of using the ophthalmic formulation according to claim 7 in a treatment and a prevention of cataract.

19. A method of using the ophthalmic formulation according to claim 8 in a treatment and a prevention of cataract.

20. A method of using the ophthalmic formulation according to claim 9 in a treatment and a prevention of cataract.
